(19) United States
(12) Patent Application Publication
(10) Pub. No.: US 2020/0400976 A1
(43) Pub. Date: Dec. 24, 2020

(54) HANDS FREE MEDICAL LIGHT WITH SELECTIVE INTENSITY

(71) Applicant: Jelani Winslow, Alhambra, CA (US)

(72) Inventor: Jelani Winslow, Alhambra, CA (US)

(21) Appl. No.: 16/905,017

(22) Filed: Jun. 18, 2020

Related U.S. Application Data
(60) Provisional application No. 62/865,376, filed on Jun. 24, 2019.

(51) Int. Cl. G02C 11/04; F21V 23/00; G02F 1/1685; G02C 7/10; A61B 90/30

(52) U.S. Cl. CPC G02C 11/04; F21V 23/003; F21W 2131/20; G02C 7/10; A61B 90/30; G02F 1/1685

(57) ABSTRACT

A hands free integrated personal task lighting system for dental or surgical use providing remote control light activation and selective adjustable light intensity output. A fixed output value light source on a headset has an electronically activated variable transmission glass which changes transparency to degrees of translucentness by varied electrical activation. The light system provides both remote light activation and illumination value control by variable control sensors in a hands-free environment.

This application claims the benefit of U.S. Provisional Application No. 62/865,376 filed on Jun. 24, 2019.

### BACKGROUND OF THE INVENTION

### 1. Technical Field

This invention relates to dental and surgical procedure lights, specifically hands-free task lighting on headsets controlled for proper light intensity.

It is understood that proper strong directed light is required for illumination of treatment areas in medical procedures or in dentistry where the patient's mouth must be illuminated to assist in the procedure required. It is also known that the intensity of such light needs to be varied for certain dental practice requirements. Medical/dental head lamp lighting systems provide a solution by directing illumination directly to the viewing area in a hands-free configuration, as needed.

### 2. Description of Prior Art

Prior art medical/dental head lamps provide for hands free task lighting source with on and off activation by switching from a remote power source thereto. Examples of such head light task lighting systems can be seen in U.S. Pat. Nos. 7,008,074, 9,968,417, re-issued patent RE46,463E and U.S. Publication 2011/0227059.

In U.S. Pat. No. 7,008,074, a hands-free control light is disclosed having a controllable light source on a pair of glasses. A light controlled by switches positioned at different angles so the operator moves their head to point at the first infrared center switch and remotely located infrared source, the first infrared switch will activate the dim light similarity the operator moves their head to point the second infrared sensor switch at the infrared source, the light will be turned on and off.

U.S. Pat. No. 9,968,417 discloses a hands-free lighting system having a master control unit, a light unit and a remote-control unit. The operator can control the head mounted light remotely through the control system.

In re-issued patent RE46,463, a remote-controlled illumination head lamp attached to a headset on a headband of the user is shown. Sensing unit within the control provides for on/off activation by detection of motion in front of the sensing unit. Illumination is increased and decreased based on power control supplied to the lamp.

### SUMMARY OF THE INVENTION

A touch free, hands free lighting system for dental or surgical use having a primary light source with a variable transparent optical lens capable of user control variation of light transmission induced by response to applied voltage defined generally as switchable glass. Control activation and degree initiation by varied sensor activation means such as motion, ambient light levels, sound and voice detection, proximity detection and/or RFID driven control sensor systems.

### DESCRIPTION OF THE DRAWINGS

FIG. 1 is a perspective graphic view of the lighting system of the invention on a headlight and magnification glasses assembly.

FIG. 2 is an enlarged side elevational view of the lighting assembly of the invention with adjustable optical switchable glass.

FIG. 3 is an enlarged cross-sectional view on lines 3-3 of FIG. 2 in non-activated form.

FIG. 4 is an enlarged cross-sectional view on lines 3-3 of FIG. 2 in activated light output form.

FIG. 5 is an enlarged side elevational view of an alternate lighting assembly for attachment to existing headlight source devices.

FIG. 6 is a partial, front elevational view of a flip down dental light filter assembly in open position.

FIG. 7 is a partial front elevational view of a flip down dental light filter assembly in closed used position.

### DETAILED DESCRIPTION OF THE INVENTION

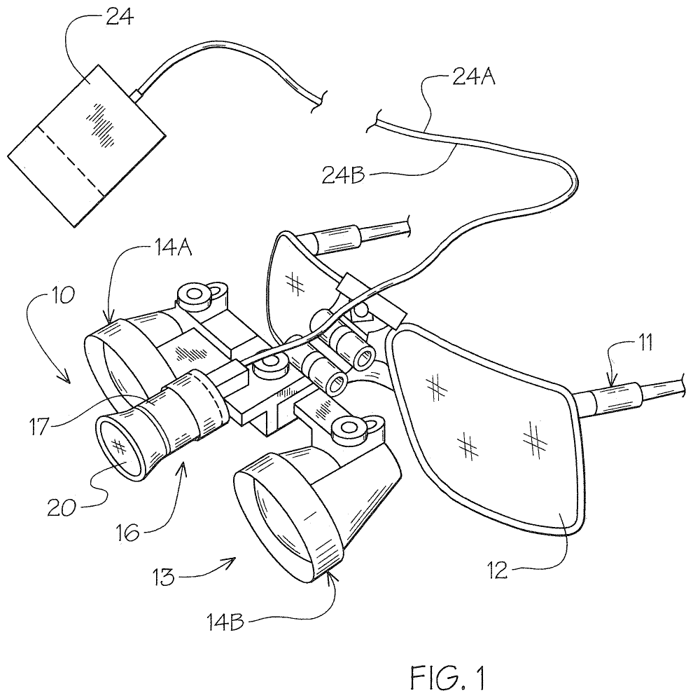

Referring to FIG. 1 of the drawings, in the example, magnifying glasses 10 can be seen with a central illuminated source light having an eyeglass support frame 11 with a pair of transparent lenses 12. A dual optical magnifying lens assembly 13 is attached centrally to the eyeglass frame 11 having a pair of spaced magnifying optics 14A and 14B on a bifurcated mounting bracket 15.

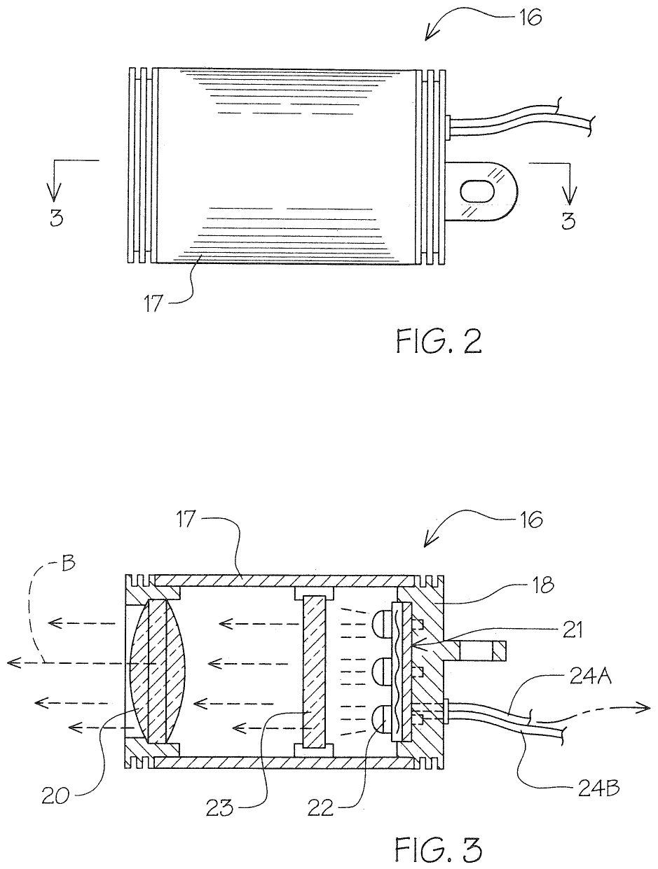

The centralized illumination source is achieved by a controllable light output lighting assembly 16 positioned centrally on the frame between the respective frame lens 12 and extending outwardly therefrom. The variable lighting assembly 16 as best seen in FIGS. 2-4 of the drawings has a cylindrical housing 17 with an annular closed base end 18 and an oppositely disposed open apertured lens mount 19. A transparent focusing lens 20 is secured within the lens mount 19 opening determining the effective projective light field therefrom. A light source defined in this example as a multiple LED light assembly 21 having a high intensity LED's 22 mounting on a support circuit board 23 is secured to the inner surface 18A of the closed base end 18 which has control and power supply access wiring openings 18B therein.

The LED lighting assembly 21 is in longitudinal alignment with the focusing lens 20 for projecting a task lighting beam B therefrom.

A remote power supply and control module 24, as best seen in FIG. 1 of the drawings, provides power and activation controller for both the LED lighting assembly 21 and a smart glass panel 23, via respective power links 24A and 24B.

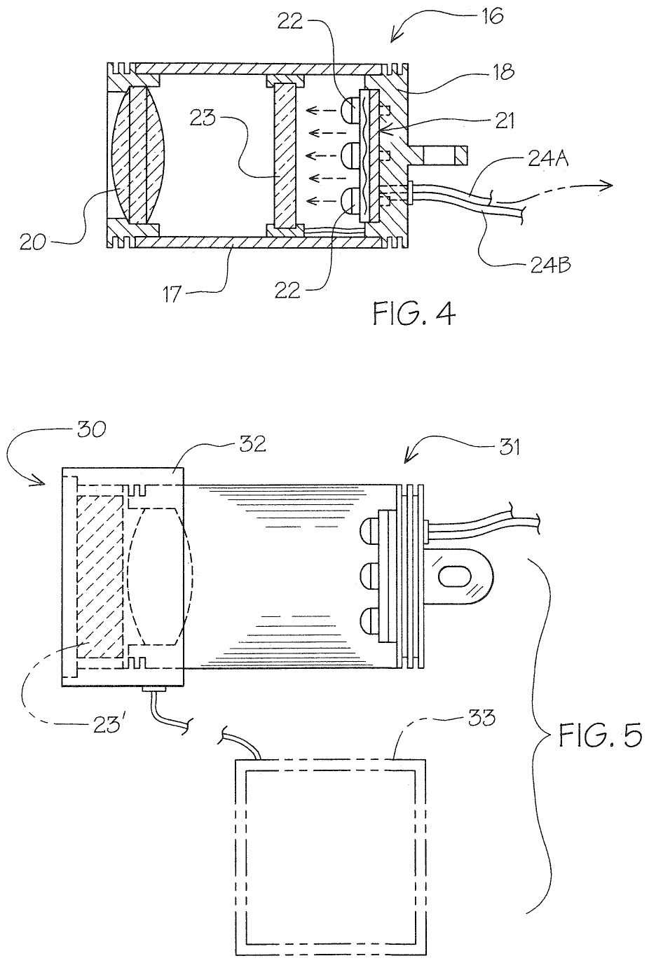

Referring now to FIGS. 3 and 4 of the drawings, the smart glass i.e. switchable glass panel 23 is mounted within the cylindrical housing 17 in longitudinally spaced relation to both the LED light assembly 21 and the transparent focusing lens 20. The switchable glass panel 23 is in electrical communication, as noted, with the power control module 24 by the respective power links 24A and 24B extending from the cylindrical housing 17. The switchable glass panel 23 is known within the art as having the ability to change its light transmission properties from transparent to opaque in response to applied voltage variations. This provides the ability to vary the light beam field transmission there through from the hereinbefore described LED light assembly 21 during operation at a constant light output.

Such switchable glass panels 23, for example, suspended particle devices SPD or polymer-dispersed liquid-crystal devices PDLCs can be used. SPD device which uses a thin film of laminate of rod-like nano-scaled particles is suspended in a liquid and placed between two pieces of glass or plastic or attached to one layer. When no voltage is applied, the suspended particles are randomly organized, thus blocking the absorbing light. When voltage is applied, the suspended particles align and let light pass. Varying the voltage of the film varies the orientation of the suspended particles thereby regulating the effective opaqueness of the glazing and the amount of light transmission.

Independent initiation of control voltage may be by any number of various activation sensors, not shown, output to the power and control module 24 which are inclusive of but not limited to motion, ambient light, sound such as voice commands and/or RFID light sensor configurations which are commercially available and well known within the art.

It will be evident from the above description that the effective lighting intensity output thereby is controlled solely by the switchable glass panel 23 so that the high intensity LED's 22 may remain constant in their effective light output during use.

As noted, the power and control module 24 can provide pre-programmed voltage control levels in response to sensor activation. A rechargeable battery drives the power and control module 24, thus providing portability to the light assembly system, in general. It will also be evident that in view of the above description that a variety of lighting assemblies 16 mounting configurations may also be used with or without the applied magnifying glass 14A and 14B used for illustration, in this example.

Referring now to FIG. 5 of the drawings, an adaptable switch glass assembly attachment 30 can be seen secured on a hands-free head light source assembly 31 representative of those available within the industry. The switch glass assembly attachment 30 has an adaptable mounting housing 32 that will be attached externally to existing headlight assemblies 31 to provide an externally mounted switchable glass panel 23′ application having the same adaptable and changeable optical qualities as hereinbefore described in the primary form of the invention's switchable glass panel 23. As before a remote power and control module 33 is needed and is shown graphically for illustration purposes in broken lines only to meet the requirement of an enabling disclosure.

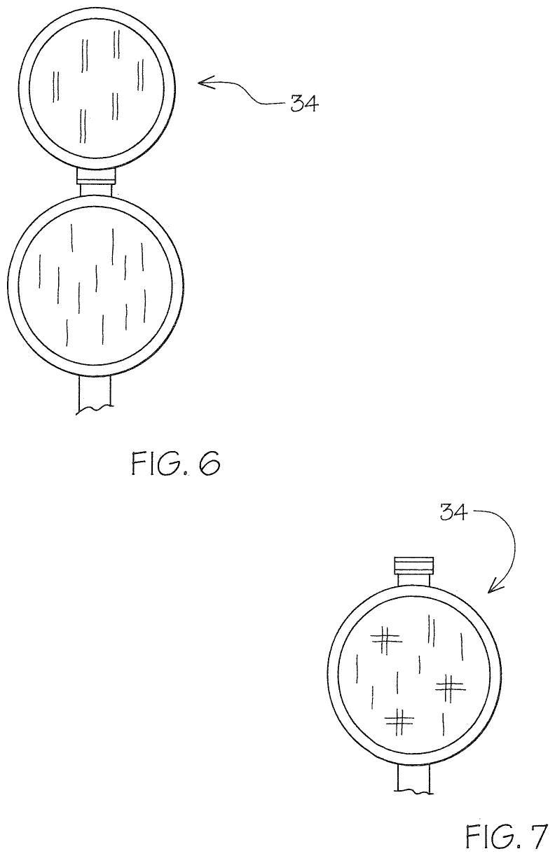

Referring now to FIGS. 6 and 7 of the drawings, other task specific attachments may be used with the hereinbefore described system of the invention such as a standard headlight flip filter 34 of a traditional orange filter medium applicable to dental filters which is flipped down to block blue light from passing from the light assembly as required during dental filling light activated material hardening parameters as an example of same.

Thus, it will be seen that a new and useful hands free medical/dental headlight lighting intensity system has been illustrated and described in both internal and external application configurations which will provide a unique and novel hands free task lighting source which intensity is controllable through the application of switchable glass 23 and 23′ which transmission qualities can be varied by pre-selected voltage inputs thereto as hereinbefore described.

Such light intensity systems have been illustrated and described herewith and it will be seen that various changes and modifications may be made thereto without departing from the spirit of the invention.

Therefore, I claim:

1. A hands free variable task lighting system for medical procedures comprises,
a control module, a light assembly, and a source of power,
said control module configured for illumination control of said light assembly by selective power supply voltage to said light assembly,
said light assembly comprising,
a light source, a selectively activated smart glass panel optical transparency responsive to varied voltage from said control module,
said smart glass panel in optical alignment with said light source, at least one user control receiving input from a user, said user control comprises at least one input device.

2. The hands-free variable task lighting system for medical procedures set forth in claim 1 wherein said control module comprises,
a power source, selective control of power to said light source and varied voltage supplied to said smart glass panel upon receiving a signal from said input device.

3. The hands-free voltage task lighting system for medical procedures set forth in claim 1 wherein said light source comprises,
at least one high intensity illumination LED in communication with said power source from said control module.

4. The hands-free variable task lighting system for medical procedures set forth in claim 1 wherein said light assembly comprises, said light source and said smart glass panel positioned within a housing having a closed base and an oppositely disposed open apertured lens mount with a transparent focusing lens secured within.

5. The hands-free variable task lighting system for medical procedures set forth in claim 1 wherein said smart glass panel optical transparency response to varied voltage comprises,
increase in optical transparency by controlled voltage applied to said smart glass panel and reduced optical transparency when no controlled voltage is applied to said smart glass panel.

6. An adaptable lighting control system for existing hands-free task lighting systems comprises,
a control module having a source of power, a light control attachment having a housing adapted to said task lighting system, a selective activated smart glass panel within said housing in optical alignment with said task lighting system optical output and in communication with said control module remotely.

7. The adaptable lighting control system for existing task lighting system set forth in claim 6 wherein said smart glass panel optical transparency increases with applied voltage from said control module and absent of controlled voltage.
